(19) United States
(12) Patent Application Publication
(10) Pub. No.: US 2010/0196352 A1
(43) Pub. Date: Aug. 5, 2010

(54) COMPOSITION FOR INCREASING STAMINA

(76) Inventors: Patrick O'Donovan, Kilkenny (IE); Colum Dunne, Co. Tipperary (IE); Joseph Murphy, Co. Tipperary (IE); Christopher Miller, Co. Laois (IE)

Correspondence Address: JACOBSON HOLMAN PLLC, 400 SEVENTH STREET N.W., SUITE 600, WASHINGTON, DC, 20004, US

(21) Appl. No.: 12/451,249

(22) PCT Filed: May 2, 2008
(86) PCT No.: PCT/IE2008/000055

§ 371 Date: Apr. 5, 2010

Related U.S. Application Data
(60) Provisional application No. 60/924,243, filed on May 4, 2007.

(51) Int. Cl. A61K 39/395; A61K 38/43; A61K 38/28; A61K 38/16; A61K 31/702; C07K 14/00

(52) U.S. Cl. 424/130.1; 424/94.1; 514/3; 514/12; 514/54; 530/350

(57) ABSTRACT

Use of whey protein isolate in the preparation of a composition for increasing mean aerobic capacity, enhanced haemoglobin production, enhanced red blood cell production and increased mean corpuscular haemoglobin concentration in competitive species, such as equine species, involved in strenuous activity. The composition may further comprise a prebiotic such as a fructo-oligosaccharide for example inulin.

The invention relates to the use of a composition for increasing stamina in competitive racing species.

### BACKGROUND OF THE INVENTION

A goal of many horse trainers has been to increase the athletic performance of horses through the implementation of nutritional and exercise strategies. Although altitude training and drugs have been used for this purpose, they have been associated with increased costs and potentially harmful side effects. As a result, these methods have limited relevance to most trainers and the use of certain performance enhancing drugs is illegal.

The oxygen carrying capacity of the blood is determined by the number of circulating functional red blood cells (Berne and Levy, 1998). Racehorses have an extremely high demand for oxygen. Horses can sequester up to 50% of their red blood cells in the spleen during times of inactivity (Persson, 1967) and release all of these cells during strenuous exercise. This physiological form of blood doping increases the horses' capacity for aerobic exercise. Small positive increases in circulating red blood cell and haemoglobin have been demonstrated to increase aerobic capacity.

In humans, the hypoxia associated with training at altitude induces a number of physiological adaptations such as increases in skeletal muscle capillary density, haemoglobin and increase of red blood cell size and possibly red blood cell numbers (Lenfant and Sullivan, 1971; Levine and Stray-Gundersen, 1997). These changes have a positive impact on aerobic capacity once the athlete returns to sea level (Dick, 1992) and may enhance aerobic ability. Similarly, in horses, training at altitude has been reported to increase total blood red cell volume and haemoglobin concentrations (Wielder and Anderson, 2000; De Aluja et al. 1968).

### STATEMENTS OF INVENTION

According to the invention there is provided use of whey protein isolate in the preparation of a composition for increasing mean aerobic capacity in competitive racing species involved in strenuous activity. In one aspect, a composition in accordance with the invention may be used to maintain the level of energy output for a longer period of time. For example, a composition in accordance with the invention may be used to improve stamina.

The competitive racing species may be an equine species. The composition may enhance haemoglobin in production in an equine species. The composition may enhance red blood cell production in an equine species. The composition may increase mean corpuscular haemoglobin concentration in equine species.

The whey protein isolate may be a digestate. The whey protein may be in an undenatured form. The whey protein isolate may contain one or more of: beta-lactoglobulin, alpha-lactalbumin, glycomacropeptides, bovine serum albumin (BSA), immunoglobulins, lactoperoxidase and lactoferrin.

The composition may further comprise a prebiotic. The prebiotic may be a fructo-oligosaccharide. For example, the prebiotic may be inulin.

The invention further provides a dietary supplement for increasing mean aerobic capacity in competitive racing species undergoing strenuous aerobic activity wherein the dietary supplement comprises whey protein isolate and a prebiotic.

The dietary compositions in accordance with the invention may enhance red blood cell and haemoglobin production and increase mean corpuscular haemoglobin concentration in competitive racing species such as equines (horses), camels, dogs, elephants, hare, kangaroo, ostrich, pigeon and birds of prey including hawks and falcons. The competitive racing species may be an equine species.

The dietary supplement may comprise 0.01% wt to 99.99% wt whey protein isolate, such as 0.01% wt to 90% wt whey protein isolate. The supplement may comprise 0.01% wt to 99.99% wt prebiotic such as 0.01% wt to 25% wt prebiotic. The dietary supplement may comprise about 88.8% wt whey protein isolate and about 11.2% wt prebiotic.

The prebiotic may be a fructo-oligosaccharide. For example, the prebiotic may be inulin.

The whey protein isolate may be obtained from the milk of a ruminant animal. Alternatively, the whey protein isolate may be obtained from equine milk.

The whey protein isolate may be a digestate. The whey protein isolate may be in an undenatured form. The whey protein isolate may contain one or more of: beta-lactoglobulin, alpha-lactalbumin, glycomacropeptides, bovine serum albumin (BSA), immunoglobulins, lactoperoxidase and lactoferrin.

In a further aspect, the invention provides a supplemented feed for increasing mean aerobic capacity in competitive racing species comprising about 10% wt whey protein isolate and about 1.25% wt prebiotic. The prebiotic may be a fructo-oligosaccharide, such as inulin. The whey protein isolate may contain one or more of: beta-lactoglobulin, alpha-lactalbumin, glycomacropeptides, bovine serum albumin (BSA), immunoglobulins, lactoperoxidase and lactoferrin.

In one aspect, the invention provides a nutritional composition that may increase red blood cell numbers and mean corpuscular haemoglobin concentration of racehorses. The invention further provides a nutritional composition that may improve the aerobic capacity of racehorses.

The nutritional composition of the invention may increase glutathione peroxidase levels and enhance glutathione concentration in racehorses.

### DEFINITIONS

Whey protein—Whey proteins are the group of globular milk proteins that remain soluble in “milk serum” or whey after the precipitation of caseins at pH 4.6 and 20° C. Whey protein is typically a mixture of beta-lactoglobulin (˜65%), alpha-lactalbumin (˜25%), and serum albumin (˜8%), which are soluble in their native forms, independent of pH. The term whey protein also includes the kappa-casein fragment (Glycomacropeptide) which remains soluble in “milk serum”.

Whey protein isolate (WPI)—is obtained by removing sufficient non-protein constituents from whey so that the finished dry product contains not less than 90% protein. WPI is produced by membrane separation processes or ion exchange.

Provon®—is a premium quality whey protein isolate derived from sweet dairy whey. The whey proteins have been extracted in a highly purified, undenatured form using cross-flow microfiltration membrane technology. Provon® is manufactured at Glanbia's designated whey processing facility in Richfield, Id., USA.

RBC—Red Blood Cell Corpuscles/Erythrocytes, cell in the blood of vertebrates that transports oxygen and carbon dioxide to and from tissues. In mammals, the red blood cell is disk-shaped and biconcave, contains haemoglobin, and lacks a nucleus. Also called erythrocyte, red cell, or red corpuscle.

Hb—Haemoglobin, the iron-containing respiratory pigment in red blood cells of vertebrates, consisting of about 6 percent heme and 94 percent globin.

PCV—Packed Cell Volume, the volume of blood cells in a sample of blood after it has been centrifuged.

MCHC—Mean Corpuscular Haemoglobin Concentration, measure of the concentration of haemoglobin in a given volume of packed red blood cell.

Prebiotic—non-digestible food ingredient that is selectively fermented by microbes of the large intestine (colon).

An “effective amount”—may be understood to mean an amount, administered at dosages and for a time, effective for achieving a desired result. Thus, an effective amount may vary depending on various factors, including but not limited to, gender, age, body type, and the desired effect.

### BRIEF DESCRIPTION OF THE DRAWINGS

A more complete understanding of the present invention and the advantages thereof may be obtained with reference to the following description and accompanying drawings wherein:

FIG. 1 illustrates a pathway for increased red blood cell production in accordance with an aspect of the invention;

FIG. 2 illustrates a pathway for increased red blood cell release from the spleen in accordance with another aspect of the invention;

FIG. 3 illustrates a pathway for increased mean corpuscular haemoglobin concentration in accordance with another aspect of the invention;

FIG. 4 illustrates a pathway for increased glutathione peroxidase production in accordance with another aspect of the invention;

FIG. 5 is a bar chart illustrating an increase in resting red blood cell concentration (RBC) in racehorses fed 500 g of whey protein isolates compared to a control group. (*=p<0.05);

FIG. 6 is a bar chart illustrating an increase in post-exercise red blood cell concentration (RBC) in racehorses fed 500 g of whey protein isolates compared to a control group (*=p<0.05);

FIG. 7 is a graph illustrating an increase in resting mean corpuscular haemoglobin concentration (MCHC) in racehorses fed 50 g of whey protein isolates in combination with 25 g of inulin compared to a control group (*=p<0.05); and

FIG. 8 is a graph illustrating an increase in post-exercise red blood cells (RBC) concentrations in racehorses fed 200 g of whey protein isolates in combination with 25 g of inulin compared to a control group (*=p<0.05).

### DETAILED DESCRIPTION OF THE INVENTION

### Whey Protein

While the physiological benefits of whey protein supplementation in human athletes are well documented little information is available on the effect of whey supplementation to horses and other competitive racing species. Whey protein supplementation has been demonstrated to be particularly beneficial in human athletes providing high biological value proteins to promote muscle growth and recovery following exercise (Rennie et al. 2000) and to provide substrates for the production of antioxidant enzymes such as glutathione (Bounous et al. 1989; Micke et al. 2002) which may help bolster immunity.

Whey proteins have a higher level of essential amino acids content when compared to various vegetable protein sources (Walzem et al. 2002) which may be found in some animal feeds. Branched-chain amino acids (BCAA) and particularly leucine, have been identified as key amino acids in protein metabolism and stimulate protein synthesis (Anthony et al. 2001). In addition, amino acid availability is a key factor in the stimulation of muscle protein synthesis (Kimball and Jefferson, 2002). Whey proteins are absorbed rapidly (Hall et al. 2003) and have been demonstrated to be a potent stimulator of skeletal muscle protein synthesis (Tipton et al, 2004) which may lead to increased skeletal muscle mass. Research in humans has demonstrated that increasing protein intake to levels above the RDA can promote positive changes in body composition i.e. increase lean tissue to fat ratio (Layman et al., 2004). As an example, US Patent Application 20030165574 to Ward discloses compositions and methods for treatment of body weight conditions.

There is some evidence to suggest that whey protein and the bioactive components in whey may enhance the immune response and reduce susceptibility to infection (Bounous, 1989; Sfeir et al 2004). Glutathione (GSH) is the main intracellular thiol antioxidant that protects against a variety of different antioxidant and participates in a number of cellular antitoxic and defensive functions. GSH acts as a scavenger of hydroxyl and carbon radicals and is important for reduction/regeneration of oxidised forms of vitamin E and C. The rate-limiting step in glutathione production is the availability of cysteine.

Whey protein is a rich source of highly bioavailable forms of the amino acid cysteine (as cystine and glutamylcysteine) from bioactive components such as lactoferrin, serum albumin and alpha-lactalbumin. Whey protein supplementation has been demonstrated to increase cellular glutathione production (Bounous et al. 1989; Bounous, 2000; Micke et al. 2002) which may bolster the antioxidant defense systems.

### Prebiotics

Prebiotics are non-digestible food ingredients that are selectively fermented by microbes of the large intestine (colon). Effective prebiotics are those which have a specific fermentation by native colonic bacteria and as a result have the ability to alter the faecal microflora composition towards a more ‘beneficial’ community structure. Both inulin and oligofructose have been demonstrated to be effective prebiotics in promoting the growth of beneficial colonic bifidobacteria and lactobacilli (Kolida S et al., 2002; Manning and Gibson, 2004).

In particular, scientific studies have shown that fructo-oligosaccharides (FOS) are specifically fermented by bifidobacteria. Ingestion of these prebiotics in humans causes bifidobacteria to become numerically dominant in faeces. Scientific data have indicated that a FOS dose of 4 g/d is prebiotic (Gibson 1998).

While preserving native colonic bacteria is important for good health, providing an ideal environment for the growth of ‘optimal’ gut microflora can increase resistance to infection by pathogenic bacteria, lower blood ammonia, increase stimulation of the immune response and reduce the risk of cancer (Manning and Gibson, 2004).

Compositions in accordance with the invention include fructo-oligosaccharides and/or inulin and combinations thereof. These compositions can be considered as prebiotic compositions which may promote the proliferation of native intestinal microflora through nutrition solutions for a subject, such as a racehorse. These compositions may be used independently or in conjunction with other nutritional supplements, such as sports nutrition products. The presence of a prebiotic in compositions in accordance with the invention may benefit the health of the large intestine (gut).

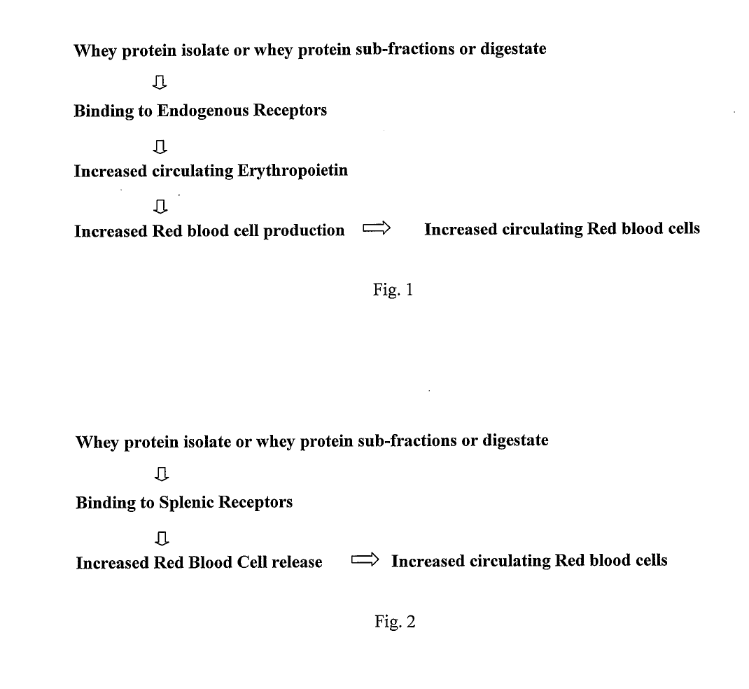

Referring to FIG. 1, whey protein isolate or whey protein sub-fractions or a digestate, such as whey protein sub-fractions produced during digestive processing, may bind to endogenous receptors and stimulate the release of erythropoietin, either directly or indirectly, resulting in increased circulating erythropoietin which may cause an increase in the production of red blood cells and haemoglobin. Referring to FIG. 2, whey protein isolate or whey protein sub-fractions or digestate may increase the level of circulating red blood cells through some splenic release mechanism during exercise. It is believed that red blood cell production may act in a dose-response manner, increasing as the concentration of whey protein or whey protein isolate sub-fractions increases.

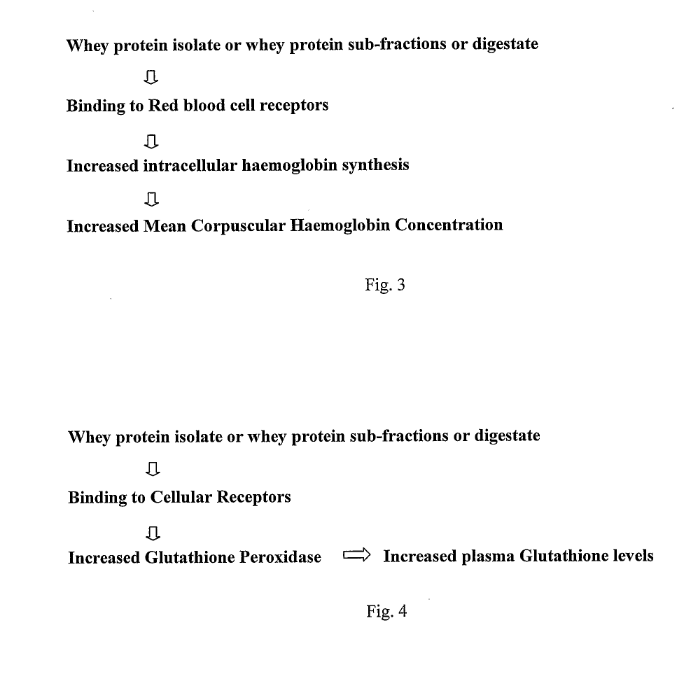

It is believed that whey protein isolate or whey protein sub-fractions or digestate may have a bioactivity regarding the increase of mean corpuscular haemoglobin concentration within the body. Referring to FIG. 3, whey protein isolate, whey protein sub-fractions or a digestate, such as sub-fractions produced during digestive processing, may stimulate red blood cell receptors, either directly or indirectly, resulting in an increase of mean corpuscular haemoglobin concentration within the body.

It is believed that whey protein isolate or whey protein sub-fractions may have bioactivity which could result in an increase in the level of Glutathione Peroxidase within the body. Referring to FIG. 4, whey protein isolate, whey protein sub-fractions or a digestate of whey protein, such as sub-fractions produced during digestive processing, may stimulate cellular receptors either directly or indirectly, resulting in an increase of Glutathione Peroxidase concentration within the body.

According to one aspect of the invention, dietary compositions and supplements that enhance red blood cell production include a whey protein isolate. The compositions and supplements of the invention may act through a pathway of increasing red blood cell production initiated and/or stimulated by whey protein isolate or whey protein sub-fractions or digestate. The whey protein isolate or whey protein sub-fraction or digestate may bind to endogenous receptors within the haematinic system, stimulating production of red blood cells and increasing haemoglobin levels. The compositions may provide increased muscle mass and strength.

Compositions and dietary supplements in accordance with various aspects of the invention may deliver effective amounts of whey protein isolates or whey protein sub-fractions or digestate to a subject. The compositions generally include whey protein isolates in an amount between about 0.01 grams and about 1000 grams. Amounts less than 0.01 grams or greater than 1000 grams may also be possible depending on the formulation. In other aspects, the amount of whey protein isolate is about 50 grams or less, about 200 grams or less, or about 500 grams or less. The whey protein isolate may contain one or more of: beta-lactoglobulin, alpha-lactalbumin, glycomacropeptides, bovine serum albumin, immunoglobulins, lactoperoxidase and lactoferrin.

Compositions and dietary supplements in accordance with various aspects of the invention may deliver an effective amount of a prebiotic such as a fructo-oligosaccharide for example inulin to a subject. The compositions generally include fructo-oligosaccharide or inulin in an amount between about 0.01 grams and about 100 grams. Amounts less than 0.01 grams or greater than 100 grams may also be possible depending on the formulation. In other aspects, the amount of fructo-oligosaccharide or inulin is about 25 grams or less.

According to another aspect of the invention, dietary compositions and supplements including whey protein isolate and in some embodiments additional components such as whey protein sub-fractions may be administrated in effective amounts such as in connection with an exercise program. The whey protein isolate may include one or more of: beta-lactoglobulin, alpha-lactalbumin, glycomacropeptides, bovine serum albumin, immunoglobulins, lactoperoxidase and lactoferrin. The compositions can be administered before, during or after exercise to enhance the effects of the exercise. Thus, methods of enhancing red blood cell and haemoglobin production, mean corpuscular haemoglobin concentration and methods of increasing muscle size and strength are also provided. Methods of supplementing a diet to enhance aerobic capacity are further provided.

The compositions can be administered to a subject one or more times per day. One or more servings are administered each time to provide an effective amount of whey protein isolate and optionally also, whey protein sub-fractions or whey peptides.

It is believed that an “effective amount” of whey protein isolate in accordance with various aspects of the invention generally deliver whey protein isolate between about 0.01 grams per kilogramme body weight of the individual and about 1.99 grams per kilogramme body weight of the subject. Amounts less than 0.01 grams per kilogramme body weight of the subject or greater than 1000 grams per kilogramme body weight of the subject may also be possible depending on the formulation and combination with other ingredients.

It is believed that an “effective amount” of prebiotic in accordance with various aspects of the invention generally deliver a fructo-oligosaccharide or inulin in an amount between about 0.01 grams per kilogramme body weight of a subject and about 100 grams per kilogramme body weight of a subject. Amounts less than 0.01 grams per kilogramme body weight of a subject or greater than 100 grams per kilogramme body weight of a subject may also be possible depending on the formulation and combination with other ingredients.

Compositions and methods in accordance with aspects of the invention also may be used in connection with other haematinic agents. For example, the compositions and methods can be used to increase oxygen delivery to the tissues.

The compositions also may include other components, such as protein, carbohydrates, fibre, fats and oils, vitamins, minerals, yeast, prebiotic, probiotic, caffeine, herbal substances and plant extracts, or other nutritional ingredients. The compositions may further include flavourings, colorants, or any other desired additives in accordance with techniques known to persons skilled in the art.

The dietary compositions in accordance with aspects of the invention may be provided in various forms, including solid form such as powder, tablets, pellets, feed cubes or capsules, or in liquid form. The powders and liquids can be administered directly, mixed with other solids or liquids, or incorporated into any number of solid and liquid food products. For example, the compositions can be formed as blended powders, granules, tablets, chewable tablets, capsules, pellets, feed cubes, and liquid syrups, gums, gels, oils to be administered directly to an individual. The dietary compositions can be administered either as part of the feedstuff, in addition to or given externally. The compositions also can be incorporated into liquid beverages or mixed with liquid beverages prior to use. Liquid beverages include water, carbohydrate syrups (e.g. molasses), acidic juice beverages (e.g., orange juice, apple juice, grape juice, grapefruit juice, cranberry juice, or blended juices), acidic beverages (e.g., sport beverages, neutral pH beverages (e.g., milk UHT dairy, RTD nutritional, soy milk, or shakes and other blended beverages such as milkshakes, smoothies, frappes), non-acidic beverages and oils (e.g. olive oil, sunflower oil, flaxseed oil, soy bean oil, wheat germ oil, corn oil, linseed oil, cotton seed oil, safflower oil, evening primrose oil, pumpkin seed oil, rice bran oil, borage oil, peanut oil, fish and marine oil). The compositions can also be incorporated into nutritional supplement foodstuffs (e.g., cereals, muesli feed mixes, energy bars or other health bars), confectionery products (e.g., chews or chewing gum), dairy products (e.g., yogurt, cheese, or processed cheese) and added to foodstuffs which contain cereals, grains, nuts or seeds.

The dietary compositions may be prepared using any conventional processing techniques, such as for example pasteurization, sterilization (canning), membrane processing, refrigeration, freezing, spray-drying, heat drying, freeze drying, vacuum drying, fluidised air drying, extrusion, mixing, irradiation, fermentation, packaging, wet, dry, fluid bed granulation, filtration.

The present invention will be more clearly understood by the following examples thereof.

### EXAMPLE 1

A typical dietary composition comprising of Provon® whey protein isolate is presented in Table 1.

TABLE 1
Typical dietary composition comprising of Provon ® whey protein isolate.

| | | |
|---|---|---|
| Calories | 370 | kcal |
| Calories from Fat | 3.8 | kcal |
| Total Fat | 0.42 | g |
| Saturated Fat | 0.25 | g |
| Polyunsaturated Fat | 0.03 | g |
| Monounsaturated Fat | 0.1 | g |
| Cholesterol | 2.0 | mg |
| Total Carbohydrate | <3.0 | g |
| Dietary Fibre | — | g |
| Sugars | 0.75 | g |
| Protein | 89 | g |
| Vitamin A | — | mg |
| Vitamin C | — | mg |
| Thiamin | — | mg |
| Niacin | — | mg |
| Riboflavin | — | mg |
| Calcium | 500-750 | mg |
| Sodium | 140-230 | mg |
| Potassium | 230-380 | mg |
| Magnesium | 80-160 | mg |
| Iron | 0.8 | mg |
| Phosphorous | 180-280 | mg |
| Chloride | <50 | mg |

### EXAMPLE 2

A typical amino acid composition of Provon® whey protein isolate is presented in Table 2.

TABLE 2
Typical amino acid composition of Provon ® whey protein isolate.

| Amino Acid | Per 100 g of formulation |
|---|---|
| Aspartic Acid | 11.9 |
| Threonine | 8.0 |
| Serine | 5.3 |
| Glutamic Acid | 18.9 |
| Glycine | 1.7 |
| Alanine | 5.3 |
| Valine | 6.3 |
| Isoleucine | 7.2 |
| Leucine | 11.2 |
| Tyrosine | 3.2 |
| Phenylanaline | 3.1 |
| Histidine | 2.1 |
| Lysine | 8.8 |
| Arginine | 2.2 |
| Proline | 7.8 |
| Cystine | 2.7 |
| Methionine | 2.2 |
| Tryptophan | 1.9 |

### EXAMPLE 3

A typical protein composition of Provon® whey protein isolate is presented in Table 3.

| | Typical Value | Range |
|---|---|---|
| Protein | (% of protein) | (% of protein) |
| Beta-lactoglobulin | 51 | 49-53 |
| alpha-lactalbumin | 23 | 20-25 |
| Glycomacropeptides | 18 | 15-20 |
| Immunoglobulins | 4 | 3-5 |
| Bovine Serum Albumin (BSA) | 2 | 1-3 |
| Lactoferrin, Lactoperoxidase, other | 2 | 1-3 |

### EXAMPLE 4

A typical dietary composition comprising a unique blend of Provon® whey protein isolate and inulin formulated as a blended dry powder is presented in Table 4 below.

TABLE 4
Typical Nutritional Information of a powdered formulation containing
88.8% Provon ® whey protein isolate and 11.2% inulin as dry powders.

| | Per 100 g | |
|---|---|---|
| Calories | 341.9 | kcal |
| Calories from Fat | 3.4 | kcal |
| Total Fat | 0.37 | g |
| Saturated Fat | 0.22 | g |
| Polyunsaturated Fat | 0.03 | g |
| Monounsaturated Fat | 0.09 | g |
| Cholesterol | 1.78 | mg |
| Total Carbohydrate | 2.1 | g |
| Dietary Fibre | 10.0 | g |
| Sugars | 1.7 | g |
| Protein | 79.1 | g |
| Vitamin A | — | mg |
| Vitamin C | — | mg |
| Thiamin | — | mg |
| Niacin | — | mg |
| Riboflavin | — | mg |
| Calcium | 446-668 | mg |
| Sodium | 129-209 | mg |
| Potassium | 205-339 | mg |
| Magnesium | 71-142 | mg |
| Iron | 0.8 | mg |
| Phosphorous | 160-249 | mg |
| Chloride | <40.0 | mg |
| Moisture | 3.7% | |
| Ash | 2.5% | |

### EXAMPLE 5

A typical amino acid composition of a formulation comprising a unique blend of Provon® whey protein isolate and inulin formulated as a blended dry powder is presented in Table 5 below.

TABLE 5
Typical Amino Acid composition of a powdered formulation containing
88.8% Provon ® whey protein isolate and 11.2% inulin.

| Amino Acid | Per 100 g of formulation |
|---|---|
| Aspartic Acid | 10.6 |
| Threonine | 7.1 |
| Serine | 4.7 |
| Glutamic Acid | 16.8 |
| Glycine | 1.5 |
| Alanine | 4.7 |
| Valine | 5.6 |
| Isoleucine | 6.4 |
| Leucine | 9.9 |
| Tyrosine | 2.8 |
| Phenylanaline | 2.8 |
| Histidine | 1.9 |
| Lysine | 7.8 |
| Arginine | 2.0 |
| Proline | 6.9 |
| Cystine | 2.4 |
| Methionine | 2.0 |
| Tryptophan | 1.7 |

### EXAMPLE 6

A typical composition of a Pelleted feed for racehorses containing 10.0% Provon® whey protein isolate and 1.25% inulin is presented in Table 6 below.

TABLE 6
A typical composition of a Pelleted feed for racehorses containing 10.0%
Provon ® whey protein isolate and 1.25% inulin.
BESTMIX - production: raw materials & nutrient
3718.0 BB TEST2 RACE CUBES

| Raw material | BBTest2 |
|---|---|
| 41 POLLARD-MENEBA | 10.00000% |
| 50 OATS | 31.73500% |
| 61 OAT POLLARD MEAL | 12.20000% |
| 70 MAIZE | 12.60000% |
| 120 BEET PULP MOLASSED | 5.00000% |
| 152 LUCERNE 16 | 5.00000% |
| 414 SOYA OIL | 3.00000% |
| 430 MOLASSES-TOTAL | 4.90000% |
| 457 INULIN | 1.25000% |
| 458 WHEY PROTEIN ISOLATE | 10.00000% |
| 512 MONO-DICALCIUM PHOSP | 1.00000% |
| 520 LIMEFLOUR | 1.00000% |
| 527 SODIUM BICARBONATE | 0.15000% |
| 529 SALT | 1.00000% |
| 530 CAL. MAG. | 0.45000% |
| 612 MYCO CURB | 0.05000% |
| 613 MYCOSORB | 0.10000% |
| 640 AGRISWEET | 0.02500% |
| 675 YEAST PREMIX | 0.04000% |
| 720 ROCHE HORSE MINS | 0.50000% |

| | | | |
|---|---|---|---|
| 2 MOISTURE | % | “ . . . ” | 11.881 |
| 4 A OIL | % | “ . . . ” | 6.152 |
| 9 C18:2 | % | “ . . . ” | 2.479 |
| 11 SAT OIL | % | “ . . . ” | 0.759 |
| 12 UNSAT OI | % | “ . . . ” | 4.208 |
| 13 *UNS/SAT | | “ . . . ”* | 5.542 |
| 35 D.E.H. | MJ/K | “ . . . ” | 10.347 |
| 40 STAR | % | “ . . . ” | 25.522 |
| 43 SUGAR | % | “ . . . ” | 5.899 |
| 44 SUGAR + ST | % | “ . . . ” | 22.330 |
| 50 PROT | % | “ . . . ” | 16.769 |
| 70 LYSINE | % | “ . . . ” | 1.149 |
| 73 METH | % | “ . . . ” | 0.329 |
| 76 THREO | % | “ . . . ” | 1.006 |
| 78 TRYPTO | % | “ . . . ” | 0.257 |
| 100 FIB | % | “ . . . ” | 9.594 |
| 101 DGF | % | “ . . . ” | 3.982 |
| 102 UDFIB RU | % | “ . . . ” | 5.732 |
| 103 NDF | % | “ . . . ” | 24.869 |
| 110 ASH | % | “ . . . ” | 6.903 |
| 111 CALCIUM | % | “ . . . ” | 0.861 |
| 112 TPHOS | % | “ . . . ” | 0.313 |
| 118 NACL | % | “ . . . ” | 1.661 |
| 119 NA | % | “ . . . ” | 0.500 |
| 120 K | % | “ . . . ” | 0.676 |
| 121 CL | % | “ . . . ” | 0.885 |
| 123 MAG | % | “ . . . ” | 0.267 |
| 124 CU | PPM | “ . . . ” | 26.134 |
| 125 CU ADDED | PPM | “ . . . ” | 20.000 |
| 126 MN | PPM | “ . . . ” | 104.420 |
| 127 I | PPM | “ . . . ” | 1.662 |
| 129 SE | PPM | “ . . . ” | 0.346 |
| 131 CO | PPM | “ . . . ” | 1.626 |
| 132 ZN | PPM | “ . . . ” | 96.181 |
| 133 SUL | % | “ . . . ” | 0.112 |
| 161 % PULP | % | “ . . . ” | 5.000 |
| 162 % BEET | % | “ . . . ” | 5.000 |
| 250 CYSTINE | % | “ . . . ” | 0.148 |
| 251 ISOLEUC. | % | “ . . . ” | 0.486 |
| 252 ARGININE | % | “ . . . ” | 0.490 |
| 253 FENYLALA | % | “ . . . ” | 0.556 |
| 254 HISTIDIN | % | “ . . . ” | 0.213 |
| 255 LEUCINE | % | “ . . . ” | 0.885 |
| 256 TYROSINE | % | “ . . . ” | 0.484 |
| 257 VALINE | % | “ . . . ” | 0.590 |
| 258 PHEN/TRY | % | “ . . . ” | 1.042 |

### Clinical Study (Horses)

Noticeable increases in the form and condition of recreational and trotting horses fed 500 g of whey protein isolates per day were observed in a feeding study. No adverse effects were observed. In particular, supplementation of feed with Provon® whey protein isolate to racehorses was shown to cause a significant increase in red blood cells and haemoglobin. This increase will improve the oxygen carrying capacity of the blood and may improve aerobic performance of race horses. It has been demonstrated in humans that even small increases in circulating haemoglobin (>5%) can improve athletic performance (Gledhill, 1982).

The effects of supplementing the present invention versus a conventional feed mix to racehorses were investigated over 63 day feeding period. The horses were fed either the present invention or conventional feed mix 3 times daily. The animals received either 0 or 500 g of whey protein isolates as part of their feed. The whey protein isolate was fed formulated as part of the coarse feed mix. Each horse was fed 8 kg of feed containing 500 g of whey protein isolate or isoenergetic control. Trial diets were balanced isoenergetically. No adverse effects were observed.

Various measurements including the analysis of blood samples were conducted on each horse (pre- and 30 minutes post-exercise) on the following days during the study 0, 21, 42, 63. Specifically, RBC, Hb, PCV, Blood Biochemistry and Antioxidant capacity were measured before and 30 minutes after exercise.

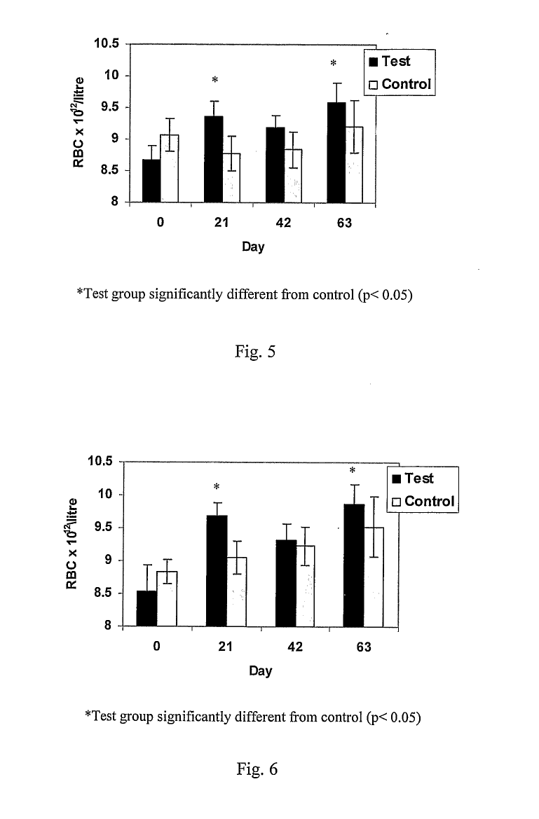

The study was conducted with 22 thoroughbred racehorses between 4-12 years of age measuring the effect of the present invention, a racehorse feed mix containing whey protein isolate compared with a placebo feed containing an equivalent amount of protein. No adverse effects were observed from increased protein intake in thoroughbred horses. The results show that whey protein isolate supplement fed mix increases the level of circulating Red Blood Cell and Haemoglobin concentrations both at rest and post-exercise and thus has the potential to enhance aerobic performance. Referring to FIG. 5, 500 g of whey protein isolates is sufficient to improve resting Red Blood Cell concentrations in racehorses. Referring to FIG. 6, 500 g of whey protein isolates is sufficient to improve post-exercise Red Blood Cell concentrations in racehorses.

The effects of dietary whey protein isolate supplementation on increasing Red Blood Cell and Haemoglobin concentration may be dose dependant. The effects of supplementing feed with whey protein isolate in a dose-response manner versus a conventional feed mix to racehorses were investigated over 63 day feeding period. The horses were fed either a supplement feed in accordance with the invention or conventional feed mix 3 times daily. The animals received either 0, 50, 200, or 500 g of whey protein isolates and 25 g of inulin daily as part of their feed. The whey protein isolate was formulated as part of a coarse feed mix. Each horse was fed 8 kg of feed containing whey protein isolate or isoenergetic control. Alternative Feeds were formulated to contain either 0%, 0.625%, 2.5% or 5% whey protein isolate and 0.3% inulin. Trial diets were isoenergetically balanced. No adverse effects were observed.

The study was conducted with 42 thoroughbred Racehorses between 4-6 years of age a Racehorse feed mix containing whey protein isolate and inulin a placebo feed containing an equivalent amounts of protein were administered to the horses. No adverse effects were observed from increased protein intake in thoroughbred horses.

Various measurements including the analysis of blood samples were conducted on each horse (pre- and immediately post-exercise) on the following days during the study 0, 21, 42, 63 and 14 days post-treatment (washout). Specifically, RBC, Hb, PCV, Blood Biochemistry and Antioxidant capacity were measured before and immediately after exercise.

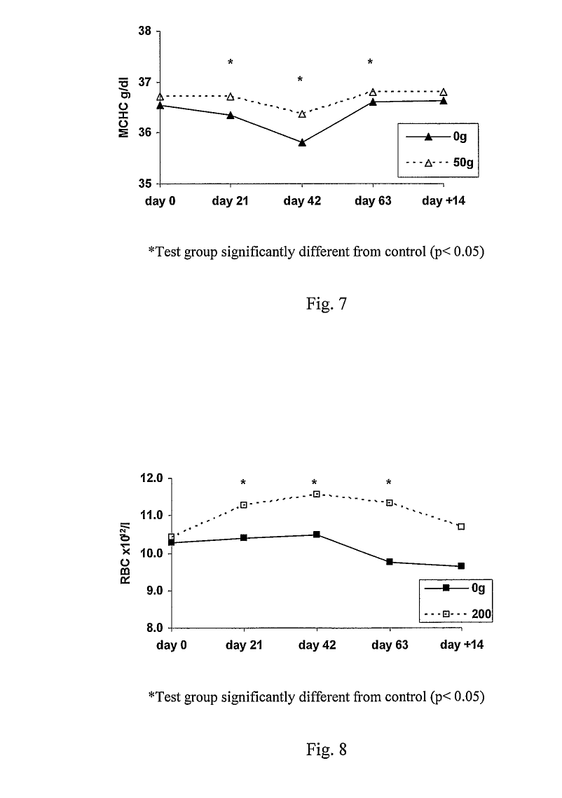

The results show that supplementary feed with whey protein isolate increases the Mean Corpuscular Haemoglobin Concentration of Racehorses. This effect will increase the oxygen carrying capacity of the blood which may improve aerobic capacity of the Racehorses. Post-exercise data suggests that supplemented feed treatment with the invention increases the level of circulating RBC during exercise and thus has the potential to enhance aerobic performance. Referring to FIG. 7, 50 g of whey protein isolates and 25 g of inulin per day is sufficient to improve Resting Mean Corpuscular Haemoglobin Concentration in Racehorses. Referring to FIG. 8, 200 g of whey protein isolates and 25 g of inulin per day is sufficient to improve Red Blood Cell numbers and Mean Corpuscular Haemoglobin Concentration in Racehorses.

The results showed that the invention increases plasma Glutathione Peroxidase of Racehorses. Increased Glutathione Peroxidase (GPx) can lead to increased Glutathione (GSH) levels and increased antioxidant capacity in Racehorses.

The invention is not limited to the embodiments hereinbefore described, with reference to the accompanying drawings, which may be varied in construction and detail.

### REFERENCES

The disclosures of the following references are incorporated herein.
Anthony J C, Anthony T G, Kimball S R, Jefferson. L S. (2001) Signaling pathways involved in the translational control of protein synthesis in skeletal muscle by leucine. J Nutr 131:1965-1972.
Berne R M and Levy M N. (1998). In: The Cardiovascular System, Physiology 4th Edition. Eds: R M Beme, M N Levy, B M Koeppen and B A Stantion, Mosby Inc pp 319-324.
Bounous G, Batist G, Gold P. (1989) Immunoenhancing property of dietary whey protein in mice: role of glutathione. Clin Invest Med 12:154-161.
Bounous G. (2000). Whey protein concentrate (WPC) and glutathione modulation in cancer treatment. Anticancer Res 20:4785-4792.
De Aluja A S, Gross D R, McCosker P J and Svendsen J. (1968) Effect of altitude on horses. Vet Rec 82:368-372.
Dick F W. (1992) Training at altitude in practice. Int J Sports Med 13:S203-S206.
Gibson G R. (1998) Dietary modulation of the human gut microflora using prebiotics. Br J Nutr. 80:S209-212.
Gledhill N. (1982) Blood doping and related issues: A brief review. Med Sci Sports Exerc 14:183-189.
Hall W L, Millward D J, Long S J, Morgan L M. (2003) Casein and whey exert different effects on plasma amino acid profiles, gastrointestinal hormone secretion and appetite. Br J Nutr 89:239-248.
Kimball S R and Jefferson L S. (2002) Control of protein synthesis by amino acid availability. Curr Opin Clin Nutr Metab Care 5:63-67.
Kolida S, Tuohy K, Gibson G R. (2002) Prebiotic effects of inulin and oligofructose. Br J Nutr. 87:S193-197.
Layman D K. (2004). Protein quantity and quality at levels above the RDA improves adult weight loss. J Am Coll Nutr 23:631 S-636S.
Lenfant C, Sullivan K. (1971) N Engl J. Med. 284:1298-1309. Adaptation to high altitude.
Levine, Benjamin D., and James Stray-Gundersen. (1997) “Living high-training low”: effect of moderate-altitude acclimatization with low-altitude training on performance. J. Appl. Physiol. 83: 102-112, 1997.
Manning T S and Gibson G R. (2004) Microbial-gut interactions in health and disease. Prebiotics. Best Pract Res Clin Gastroenterol. 18:287-298.
Micke P, Beeh K M, Buhl R. (2002) Effects of long-term supplementation with whey proteins on plasma glutathione levels of HIV-infected patients. Eur J Nutr 41:12-18.
Persson S G B. (1967) On blood volume and working capacity. Acta Vet Scand Suppl 19:1-189.
Rennie M J and Tipton K D. (2000) Protein and amino acid metabolism during and after exercise and the effects of nutrition. Annu Rev Nutr 20:457-483.
Sfeir R M, Dubarry M, Boyaka P N, Rautureau M, Tome D. (2004) The mode of oral bovine lactoferrin administration influences mucosal and systemic immune responses in mice. J. Nutr. 134:403-409.
Tipton K D, Elliott T A, Cree M G, Wolf S E, Sanford A P, Wolfe R R. (2004) Ingestion of casein and whey proteins result in muscle anabolism after resistance exercise. Med Sci Sports Exerc. December; 36:2073-2081.
Walzem R L, Dillard C J, German J B. (2002) Whey components: millennia of evolution create functionalities for mammalian nutrition: what we know and what we may be overlooking. Crit Rev Food Sci Nutr. 42:353-375.
Wickler S J and Anderson T P. (2000) Hematological changes and athletic performance in horses in response to high altitude (3,800 m). Am J Physiol Regulatory Integrative Comp Physiol 279:R1176-R1181.

1-29. (canceled)

30. A use of whey protein isolate in the preparation of a composition for increasing mean aerobic capacity in competitive racing species involved in strenuous activity.

31. The use as claimed in claim 30 wherein the racing species is an equine species.

32. The use as claimed in claim 31 wherein the composition enhances haemoglobin production in an equine species.

33. The use as claimed in claim 31 wherein the composition enhances red blood cell production in an equine species.

34. The use as claimed in claim 31 wherein the composition increases mean corpuscular haemoglobin concentration in equine species.

35. The use as claimed in claim 30 wherein the whey protein isolate is a digestate.

36. The use as claimed in claim 30 wherein the whey protein isolate is in an undenatured form.

37. The use as claimed in claim 30 wherein the whey protein isolate contains one or more of: beta-lactoglobulin, alpha-lactalbumin, glycomacropeptides, bovine serum albumin (BSA), immunoglobulins, lactoperoxidase and lactoferrin.

38. The use as claimed in claim 30 wherein the composition further comprises a prebiotic.

39. The use as claimed in claim 38 wherein the prebiotic is a fructo-oligosaccharide.

40. The use as claimed in claim 38 wherein the prebiotic is inulin.

41. A dietary supplement for increasing mean aerobic capacity in competitive racing species undergoing strenuous aerobic activity comprising whey protein isolate and a prebiotic.

42. The dietary supplement as claimed in claim 41 wherein the racing species is an equine species.

43. The dietary supplement as claimed in claim 41 comprising 0.01% wt to 99.99% wt whey protein isolate.

44. The dietary supplement as claimed in claim 41 comprising 0.01% wt to 90% wt whey protein isolate.

45. The dietary supplement as claimed in claim 41 comprising 0.01% wt to 99.99% wt prebiotic.

46. The dietary supplement as claimed in claim 41 comprising 0.01% wt to 25% wt prebiotic.

47. The dietary supplement as claimed in claim 41 comprising about 88.8% wt whey protein isolate and about 11.2% wt prebiotic.

48. The dietary supplement as claimed in claim 41 wherein the prebiotic is a fructo-oligosaccharide.

49. The dietary supplement as claimed in claim 41 wherein the prebiotic is inulin.

50. The dietary supplement as claimed in claim 41 wherein the whey protein isolate is obtained from the milk of a ruminant animal.

51. The dietary supplement as claimed in claim 41 wherein the whey protein isolate is obtained from equine milk.

52. The dietary supplement as claimed in claim 41 wherein the whey protein isolate is a digestate.

53. The dietary supplement as claimed in claim 41 wherein the whey protein isolate is in an undenatured form.

54. The dietary supplement as claimed in claim 41 wherein the whey protein isolate contains one or more of: beta-lactoglobulin, alpha-lactalbumin, glycomacropeptides, bovine serum albumin (BSA), immunoglobulins, lactoperoxidase and lactoferrin.

55. A supplemented feed for increasing mean aerobic capacity for competitive racing species comprising about 10% wt whey protein isolate and about 1.25% wt prebiotic.

56. The supplemented feed as claimed in claim 55 wherein the prebiotic is a fructo-oligosaccharide.

57. The supplemented feed as claimed in claim 55 wherein the prebiotic is inulin.

58. The supplemented feed as claimed in claim 55 wherein the whey protein isolate contains one or more of: beta-lactoglobulin, alpha-lactalbumin, glycomacropeptides, bovine serum albumin (BSA), immunoglobulins, lactoperoxidase and lactoferrin.
